(19) United States
(12) Patent Application Publication
(10) Pub. No.: US 2007/0077555 A1
(43) Pub. Date: Apr. 5, 2007

(54) Adsorption system for the removal of viruses and viral components from fluids, in particular blood and blood plasma

(75) Inventors: Gotz Nowak, Erfurt (DE); Elke Bucha, Erfurt (DE); Ute Lange, Kahla (DE); Sven Zeeb, Magdala (DE); Alrun Schumann, Jena (DE)

Correspondence Address: KAGAN BINDER, PLLC, SUITE 200, MAPLE ISLAND BUILDING, 221 MAIN STREET NORTH, STILLWATER, MN, 55082, US

(73) Assignee: HaemoSys GmbH

(21) Appl. No.: 11/326,639

(22) Filed: Jan. 6, 2006

(30) Foreign Application Priority Data
Oct. 1, 2005 (DE) 102005001162.4-55

(51) Int. Cl. C12Q 1/70; C12Q 1/68

(52) U.S. Cl. 435/5; 435/6

(57) ABSTRACT

The present invention relates to an adsorption system for the removal of viruses and viral components, in particular hepatitis C viruses, from physiological fluids, in particular whole blood or blood plasma, by means of extracorporeal adsorption processes, as well as to an adsorber material for use in the adsorption system of the present invention.

The present invention relates to an adsorption system for the removal of viruses and viral components, in particular hepatitis C viruses, from physiological fluids, in particular whole blood or blood plasma, by means of extracorporeal adsorption processes, as well as to an adsorber material comprising porous polymer particles for use in the adsorption system of the present invention.

It is a known fact that viral infections constitute a significant health risk. In many cases, the prophylactic and therapeutic treatments presently available in this area remain completely unsatisfactory.

More than 200 million people worldwide suffer from hepatitis C alone. At the present time, there is no prophylactic vaccine protection against this and other prevailing infectious diseases, such as e.g. acquired immunodeficiency syndrome (AIDS).

The combination treatment with PEG-interferon and ribavirin, which is currently used as the standard therapy for treating hepatitis C, is only successfully in about 50 to 60% of the patients, as well as time-consuming and expensive. Due to its large number of potential side-effects, many patients discontinue the treatment or it is already contra-indicated from the beginning.

In view of the significance of viral infections for public health policies worldwide, there is a considerable scientific and commercial interest in the development of new antiviral treatments. This interest is not limited to treatment methods which allow a therapy of viral infections in a patient, but also extends to processes by means of which the transmission of viral infections via blood or plasma donations can be prevented.

New drugs against hepatitis C, such as e.g. nucleoside analogues, inhibitors of virus-specific enzymes or modulators of the immune reaction, are still in the early stages of clinical trials (Dev. A. et al.: Infect. Med. 21:28-36, 2004). The rapid occurrence of development of resistance, which is due to the high mutation rate in the virus genome, is the main limiting factor for treatments. Furthermore, at this time no definite predictions can be made regarding the effectiveness and safety of the new treatment approaches. Particularly in the case of the long-term treatment of hepatitis C, there is the danger of severe side-effects such as carcinogenicity and organ toxicity.

Adsorptive extracorporeal treatment processes are currently used for example in the therapy of severe forms of hypercholesterolaemia as well as severe auto-immune diseases such as rheumatoid arthritis and idiopathic thrombocytopenic purpura. In these cases, the adsorption of lipoproteins, immunoglobulins or immune complexes preferably takes place via surface-bonded ligands such as protein A, antibodies or negative charges of the adsorber surface. Due to the poor cell compatibility of the adsorber materials, most of these processes can only be carried out in blood plasma and not in whole blood.

Numerous approaches have been made to broaden the application possibilities of extracorporeal treatment processes. Special emphasis is given to research aimed at the removal of other pathogenic macromolecules, e.g. from the areas of toxins, antibodies, immune complexes or cytokins. Such processes have also been recognized as a way of removing pathogens from fluids. In this connection, previously non-therapeutic processes have been used which remove pathogens in vitro from blood donations or plasma donations. Filtration processes are preferably used for this purpose. For instance, EP 002 949 and EP 0 517 501 disclose the use of specially structured porous polymeric membranes to filter viruses out of different protein-containing fluids. EP 1 444 996 describes a virus filter which allows the simultaneous removal of viruses and leucocytes; in addition to filtration, its effectiveness is based on an activation of the complement systems in the blood samples initiated by the membrane surface. For use in vitro, cell receptors were also stabilized for viruses in such a manner as to allow their surface fixation to act as a virus adsorber for contaminated fluids (WO89/01813). These processes are not intended to be used directly in a patient.

In contrast to the in vitro processes for the decontamination of fluids, blood plasma or blood wherein filtration processes take center stage, the goal is to exploit extracorporeal processes for a therapeutic use against viral infections mainly on the basis of adsorptive techniques. Here, the emphasis is preferably placed on specific interactions with special recognition structures on the virus surface which, however, entail the inherent problem of the occurrence of acquired resistance. For example, it has been suggested to remove viruses by adsorption on surface-bonded monoclonal or polyclonal antibodies (DE 101 18 115, U.S. Pat. No. 6,528,057) or parts of antibodies (CN 1457899) which are directed specifically to structures on the virus surface. WO2004/064608 discloses establishing the adsorption via the affinity of certain lectins to proteins of the virus coat (GP120 of the human immunodeficiency virus (HIV)). The bonding of HIV to a surface-fixed Cl esterase inhibitor (EP 0 966 976) or bonded CD4 cell receptors for HIV (U.S. Pat. No. 6,174,299) have been described as well.

Unspecific processes for virus adsorption use chemically modified polymer surfaces, e.g. sulfate groups (EP 0 679 436), or slightly acidic or basic groups (U.S. Pat. No. 5,041,079), as are for example present in ion-exchange resins. However, in these cases, undesirable interactions with blood or plasma components often occur upon contact with whole blood. This limits the long-term use of such adsorber materials since their surface chemistry promotes activation processes such as blood coagulation which renders the adsorber device impermeable after only a short period of use. Accordingly, a direct contact of the adsorber material and the cellular components of whole blood has to be avoided, e.g. by means of prior filtration, thus separating blood components such as erythrocytes, leucocytes and thrombocytes and leaving the plasma. The process disclosed in WO01/40448 for an elimination of viruses by means of filtration through membranes having polymer side chains of a certain thickness and length and with various functional groups on their surface is not suitable for a therapeutic use in whole blood, either.

An unspecific reduction of the viral load in the blood of patients infected with hepatitis C viruses (HCV) is described in some extracorporeal processes used in connection with different indications. For example, a reduction of the viral load occurs during a cell apheresis process which is used in the treatment of ulcerative colitis and removes granulocytes and monocytes by adsorption to rough cellulose particles. While Sawada et al. only describe a limited additional reduction of the HCV-RNA in Therapeutic Apheresis 7: 547-553, 2003, they disclose a therapeutic effect upon increase of the treatment frequency in U.S. Pat. No. 6,713,252.

On average, hepatitis C patients who are treated regularly with an artificial kidney showed a lower viral load than hepatitis C patients who did not undergo dialysis. However, a direct removal of viruses due to the membrane of certain dialysis units remains controversial (Fabrizi Fet et al.: J. Nephrol 16: 467-475, 2003). The data regarding a possible reduction of the viral load in the blood of patients infected with HCV in connection with lipid apheresis processes is also contradictory (Marson, P. et al.: Int. J. Artif. Organs 22: 640-644, 1999; Schettler V. et al.: Eur. J. Clin. Invest. 31: 154-155, 2001). No therapeutic effects with respect to the pre-existing viral infections have been shown for any of these processes.

The current state of the art gives rise to the need for an efficient, simple, well-tolerated and economically feasible process for the specific removal of viruses from the blood or blood plasma of patients infected with the virus, without any development of resistance and, together with the body's own defenses, leading to a cure of the viral infection.

It is therefore the object of the present invention to develop an alternative possibility of controlling and preventing viral infections which avoids the disadvantages of the antiviral treatment approaches of the prior art particularly with respect to effectiveness, development of resistance and toxicity at as low a cost as possible. The treatment option should be applicable to viral infections which are accompanied by long-term viremia. It was the particular goal of the present invention to provide an adsorption system using a blood-compatible adsorber material which in view of the specific treatment approach is suitable for purposefully removing viruses and viral components, however, whose mechanism is not geared towards only one or a few viral surface structures.

This object is achieved by an adsorption system for the removal of viruses and viral components from physiological fluids, in particular blood or blood plasma, comprising a particulate adsorber material. The adsorber material comprises particles with a diameter of 20 to 500 μm having a polymer matrix with throughpores.

Depending on the type of virus, pore radii between 25 and 1,000 nm have been found to be especially suitable for the removal of viruses.

The adsorption takes place while the fluid, such as virus-containing whole blood or blood plasma, is in contact with the adsorber material which can for example be provided in the form of a filling of a cartridge or a cartridge-type receptacle. The decreased viral load resulting from the use of the adsorption system of the present invention constitutes the treatment and prophylaxis of viral infections.

In order to obtain a reduction in the number of viruses in the organism of infected patients, the adsorption system is preferably brought into contact with the blood or blood plasma of the patient using an extracorporeal process. For example, it can be integrated into the blood or plasma circulation by means of an extracorporeal circulation. However, the system according to the present invention is also suitable for treating physiological fluids which are not or not directly reintroduced into the patient's organism, such as e.g. blood donations or plasma donations.

It has surprisingly been found that polymer particles with special porous properties are capable of adsorbing viruses in such a manner that they can be removed irreversibly from an aqueous solution, blood plasma or blood. The use of porous particles for the removal of different pathological substances or toxins in the range of small and medium molecule sizes up to about 30,000 dalton from blood plasma or blood by means of extracorporeal therapeutic processes is disclosed in EP 1 115 456. However, so far there has been no indication that by means of porous, particulate adsorption materials such complex biological structures like viruses can efficiently be removed even from whole blood without the use of biological recognition structures such as for example surface-bonded antigens or antibodies.

The particles of the adsorber material are particles with a polymer matrix interspersed with throughpores. Such throughpores run through the polymer matrix in the form of channels which inside the particle can run separately, branch out and/or cross each other. In view of the flow behavior of the fluid to be treated, branched and/or crossing channels are preferred. The throughpores are open towards the particle surface in such a manner that a permeation of the particles is guaranteed. However, the particles of the present invention should not be microcapsules with a porous surface, i.e. particles wherein a relatively thin shell (up to about 5% of the total diameter) encloses a hollow core not containing any matrix material.

The term “pore radius” as used in the present invention refers to a pore width of cylinder-shaped channels as an average of all pores and their lengths as it can be determined in a model with a standardized measuring method (inverse size-exclusion chromatography). Accordingly, depending on the material to be removed, the pore radius is preferably between 25 and 1,000 nm, especially preferred between 50 and 600 nm. For most viruses, a radius of 80 to 450 nm is used.

It has been found that for an effective removal of viruses, the pore radius is preferably larger than the radius of the virus. For the hepatitis C virus, which has a diameter of 40 to 75 nm, the preferred pore radius is 80 to 450 nm.

For an efficient separation of viruses and viral components, the porosity of the particles (expressed as the ratio of the pore volume to the total volume of the matrix and the pores contained therein) is preferably between 0.25 and 0.75, more preferred between 0.4 and 0.6.

The adsorption system of the present invention comprises particles with an average diameter of 20 μm or more, preferably 50 μm or more, e.g. 80 μm or more or 100 μm or more. The upper limit of the average diameter is 500 μm, preferably 350 or 250 μm. Particles with an average diameter of 100 to 250 μm are especially preferred. The particles preferably have a spherical shape.

The preferred particle size guarantees that sufficiently large spaces are present between the particles of the adsorption material in the adsorption system of the present invention. This is e.g. important in the treatment of whole blood or other heterogeneous fluids since large-size blood components such as erythrocytes, leucocytes and thrombocytes need these spaces to pass through the adsorption system while the plasma together with the viruses preferably flows through the throughpores of the particles. By selecting the particle size accordingly, these spaces should preferably be adjusted such that the above-mentioned cellular blood components can just pass through them and that their passing creates a flow resistance which guarantees a transfer of the virus-containing plasma through the pores.

The specific surface of the particles of the adsorption material is usually 50 to 1,000 m²/g, preferably 50 to 500 m²/g, especially preferred 50 to 300 m²/g or 50 to 150 m²/g.

The porous particles present in the adsorption system comprise a polymer matrix; this term encompasses also copolymers of two or more different monomers. The particles used in the present invention are not restricted with respect to suitable polymers, as long as the polymer material does not release incompatible products upon contact with physiological fluids or is itself incompatible with the fluid at issue, and is suitable for providing a porous matrix. In order to impede an unspecific binding of e.g. protein components such as fibrinogen or of cellular components such as thrombocytes to the particle surface, the polymers used in the matrix preferably contain no groups which are present in ionic form or can be ionized under physiological conditions.

The materials used for the porous polymer matrix of the particles of the adsorber material should preferably exhibit sufficient rigidity so that the particles are not deformed by the pressure of the fluid passing through them. The polymer matrix has to be dimensionally stable in aqueous solutions, i.e. the particle or pore size should not change due to a swelling of the particles.

Examples of suitable polymers to provide the porous polymer matrix of the particles of the adsorber material include vinyl compounds, preferably methacrylic acid esters or styrenes, or copolymers thereof with suitable cross-linking agents, such as ethylene glycol dimethacrylate or divinyl benzene.

In view of a possibly necessary additional treatment of the polymer matrix with an agent that imparts additional hemocompatibility to the particle surface, polymers as described in EP 0 975 680 or EP 1 282 695 are preferably used in the porous polymer matrix. For their preparation, a type of monomer is used which in addition to a polymerizable double bond or a polycondensable functional group comprises a further carbonyl group in the form of a ketone or a carboxylic acid derivative which does not participate in the polymerization reaction. Preferably, the polymer comprises structural elements of the formula (A): 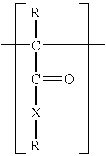
wherein the groups R can be the same or different and represent an alkyl or aryl group or a hydrogen atom. The alkyl groups can be linear, branched or cyclic and preferably consists of 1 to 20 carbon atoms, especially preferred 1 to 8 or 1 to 4 carbon atoms. The aryl group preferably consists of 6 to 18, especially preferred 6 to 12 carbon atoms. The bivalent group X is optional and represents O, NR or CH₂, wherein R is as defined above. However, as stated above, those representatives of structural unit (A) are preferred which are not present in ionic form or cannot be ionized under physiological conditions. Thus, an alkoxy group is an especially preferred group -X-R, wherein its alkyl portion is as defined above and preferably a methyl group.

Especially preferred monomers for providing structural elements (A) are alkyl methacrylates with an alkyl group preferably comprising 1 to 6 carbon atoms, such as e.g. methyl methacrylate, ethyl methacrylate or propyl methacrylate. Furthermore, vinyl acetate, cyclohexyl methacrylate or phenyl methacrylate or mixtures of the above-mentioned monomers can be used. However, for the polymer matrix of the particles of the adsorber material a polymer comprising units derived from methyl methacrylate as a monomer is especially preferred.

Copolymers or polymer mixtures containing any proportions of the above-mentioned polymers with structural elements of formula (A) or containing one or more additional polymer component(s), for example polystyrene, polyacrylonitrile or polyamides can also be used. Preferably, the monomers which provide structural element (A) after polymerization are present in an amount of at least 5 mol-%, for example 10 or 20 mol-%, in the total amount of monomers. It is especially preferred that this amount be at least 30 mol-%, and particularly preferred at least 50 mol-%.

The porous polymer matrix preferably comprises cross-linked polymers. For their production, crosslinking agents, i.e. network-forming bi- or multifunctional monomers, are used together with the above-mentioned monomers, which bi- or multifunctional monomers comprise for example two or more polymerizable groups and are added during polymerization. Purely illustrative examples of such monomers include divinyl benzene, ethylene glycol dimethacrylate or butylene glycol dimethacrylate together with suitable network-forming solubilizers. Polymethyl methacrylate co-ethylene glycol dimethacrylate is an especially preferred copolymer for providing the porous particles. The molar ratio of methyl methacrylate and ethylene glycol dimethacrylate is preferably 4:2 to 0.25:1 and especially preferred 2:1 to 1:2.

Particles with a polymer matrix with the required particle and pore sizes can be produced according to known processes. Suitable processes include for example suspension polymerization wherein a first monomer, optionally a cross-linking agent and a second (outer) phase are required, and the monomer and cross-linking agent must not be soluble in the outer phase. The free radical used as initiator is dissolved in the monomer. The monomer mixture is finely dispersed in the outer phase (e.g. water) when subjected to mechanical energy, most expediently stirring. The addition of water-soluble protective colloids prevents the monomer phase from coalescing and breaking. During the suspension polymerization, spherical polymer beads are formed whereby the size of the individual particles can largely be adjusted by varying the preparation conditions such as the stirring speed and the type and amount of protective colloid. By adding so-called porogens, which influence the mixing thermodynamics during polymerization, it is possible to incorporate pores into the material in a calculated manner. The basic prerequisite for this is that the polymer becomes insoluble in its monomer as the reaction progresses and precipitates. This can for example be achieved by the addition of a cross-linking monomer. The addition of an inert swelling agent allows a purposeful postponement of the gel point and thus the precipitation point, which makes it possible to adjust the pore size to the desired value. Another possibility of influencing the material is the addition of an inert precipitating agent. The porosimetric parameters were determined by means of inverse size-exclusion chromatography (in this connection, see Gorbunov, A et al.; J Chromatogr 448, 307 (1988)). As regards the preparation of porous particles, cf. Seidl, J., Malinstky, J.; Dusek, K; Heitz, W.; Adv. Polym. Sci.; 5; 113-213 (1967); Logemann, H. et al.; in: Houben/Weyl; Methoden der Organischen Chemie [methods in organic chemistry]; Vol. 14; 133-438 (1967); Polymerisation der Acryl- und Methycrylsäure, ihrer Salze, Halogenide und Anhydride [polymerization of acrylic and methacrylic acid, their salts, halides and anhydrides]; Volker, T. et al.; ibid; 1018-1072; Synthesis and characterization of porous polymers; Sherrington, D.C.; Makromol. Chem., Macromol. Symp.; 70/71, 303 (1993); Porous Poly-HEMA beads prepared by suspension polymerization in aqueous medium; Horak, D.; Ledniki, F.; Rehak, V.; Svec, F.; J. Appl. Polym. Sci.; 49, 2041 (1993); Chromatographic properties of macroporous beads from poly (GMA-co-EDMA); Horak, D.; Svec, F.; Tennikowa, T. B.; Nahunek, M.; Angew. Makromol. Chem.; 195, 139 (1992); Dispersion copolymerization of methyl methacrylate and acrylic acid in polyr media; Huang, J. X.; Yuan, X. Y.; Yu, X. L.; Zhang, H. T.; Polym. Int; 52, 819 (2003); Preparation and porosity characterization of highly cross- linked polymer resins derived from multifunctional methacrylate monomers; Rohr, T.; Knaus, S.; Grber, H.; Sherrington, D. C.; Macromolecules; 35, 97 (2002); Preparation by suspension polymerization of porous beads for enzyme immobilization; Skovby, M. H. B.; Knops, J.; J. Appl. Polym. Sci.; 39, 169 (1990); Reactive polymers XXXIII; The influence of the suspension stabilizer on the morphology of a suspension polymer; Horak, D.; Pelzbauer, Z.; Svec, F.; Kalal, J.; J. Appl. Polym. Sci.; 26, 3205 (1981); New designs of macroporous polymers and supports; from separation to biocatalysis; Svec, F.; Frechet, J. M. J.; Science; 273, 205/1996).

By applying surface-passivating agents such as inhibitors of the thrombocyte or cell activation to the particle surface, the hemocompatibility of the particles can be increased even further and/or the virus-adsorbing capacity can be boosted, in particular in the case of contact with blood as the preferred fluid to be treated. When used, such agents are preferably applied to the external and internal surfaces (i.e. the internal surfaces of the pores) of the porous polymer matrix. The term haemocompatibility and agents for improving it are known to the person skilled in the art, cf. e.g. Vienken J: Biological processes related to blood-biomaterial interaction; in: Blood-Material Interaction, International Faculty For Artificial Organs (INFA), Glasgow, Krems, 1998.

Suitable processes known from the prior art include: Applying high-molecular poly(N-trifluoroalkoxy)phophazene, treating the porous particles with a phophazene solution in an organic solvent and evaporating the solvent, or electrostatically or covalently bonding suitable surface-passivating agents, such as e.g. heparin, to appropriately functionalized surfaces.

However, within the framework of the present invention, hemocompatibility-improving agents are preferably applied to the porous polymer matrix with the help of the interaction between certain polymers and special linkers as it is described in EP 0 975 680 and EP 1 282 695. EP 1 282 695 already discloses hemocompatible polymer surfaces which are suitable for providing hemocompatible particles for the present invention. According to that document, porous polymer particles comprising the above-described structural elements (A) and especially preferred units derived from alkyl methacrylates are brought into contact with agents that improve hemocompatibility and carry a linker as described in the above-mentioned publications.

Polyalkylene glycols, polyalkylene imines, polyalkylene amines or polyalkylene sulfides as well as polyoxazillines are preferably used as linkers, with polyalkylene glycols being especially preferred. The average degree of polymerization of these polymeric linkers is preferably below 300, especially preferred below 150. The lower limit is usually 5, preferably 10, whereby the preferred degrees of polymerization can vary within the above-mentioned ranges based on the selection of the basic repeating units of the linker. It is especially preferred that polyethylene glycols (PEG) be used as linkers. In order to guarantee optimum stability, they are covalently linked with the agent improving hemocompatibility. The direct contact of the linker-bonded agent with a polymer surface exhibiting the necessary structural element results in a linker-mediated bond between agent and polymer which is highly stable under physiological conditions.

Thus, the present invention can preferably make use of the technological possibilities disclosed in EP 0 975 680 and EP 1 282 695 for preventing an interaction between the particle surface and blood components while at the same time maintaining or increasing the virus adsorption capacity. Based on the findings reported in EP 0 989 856 and EP 1 282 695, linker-coupled polyorganosiloxanes and other substances improving the hemocompatiblity of the surfaces are fixed to the particle surface in a predetermined concentration during a simple incubation process. Particularly suitable in this context are polyorganosiloxanes which, after being chemically bound to certain linkers (e.g. PEG), are water soluble preferably at 20° C. in amounts of 0.1 μg/ml to 100 mg/ml, preferably 1 mg/ml to 100 mg/ml or to 10 mg/ml.

Suitable hemocompatibility-improving substances include for example cholesterol or polydimethyl siloxanes which are coupled with one of the above-mentioned linkers such as e.g. PEG, or a copolymer of PEG and polypropylene glycol. Furthermore, PEG or PEG/polypropylene glycol copolymer-containing substances, such as PEG derivatives of fatty acid esters of multivalent alcohols (e.g. PEG glycerin fatty acid ester), PEG sorbitan fatty acid esters (e.g. tween products), PEG fatty acid esters, PEG lipids or PEG proteins (e.g. PEG albumin) can also be used as hemocompatibility-improving substances. Also, anticoagulative agents which specifically reduce the thrombogenity of the surfaces such as for example PEG thrombin inhibitors can be used to improve hemocompatibility.

For an efficient removal of viruses or viral components, the porous particles described herein do not require any recognition structures immobilized on their surface. Prior to their contact with the fluid to be treated, they are preferably essentially free of proteins or nucleic acids which can promote a bonding of viruses or viral components. In this context, the expression “essentially free” means that possible impurities/residues of such biological molecules do not contribute to the adsorption of the viruses or viral components. The particles can also be completely free of proteins and nucleic acids.

The adsorption system of the present invention preferably comprises a rigid or flexible housing through which the fluid to be treated can flow and which contains the above-described porous particles as adsorber material. The porous particles in the housing should preferably not be linked to each other but merely be present as loose particles. It should be ensured that the passing fluids flow around the particles evenly. The geometry and material of the housing are not restricted as long as their hemocompatibility is kept in mind. For example, the housing can be a cartridge. It may have any shape, and can, for example, be cylindrical. In order to ensure a continuous passage, the housing preferably comprises an inlet which allows the entrance of fluids, in particular body fluids, such as blood or plasma, into the housing and ensures the contact of the fluids with the polymer particles, and an outlet which allows the flowing off of the fluid after the contact with the polymer particles. The system furthermore preferably comprises one or more devices suitable for preventing the particles from exiting the housing, such as e.g. filters or frits. They keep the particles in the housing and ensure at the same time an unimpeded passage of the fluids to be treated. The system can be set up such that it can be integrated into an extracorporeal circulation.

The adsorption system of the present invention can be integrated into the blood or plasma circulation by means of an extracorporeal circuit. However, it is also suitable for treating blood donations or plasma donations or other physiological fluids withdrawn from an organism but not or not immediately returned to it after passing through the unit. The system of the present invention can be used alone or in combination with other units such as e.g. hemodialysis units. The flow of the fluids through the system can be effected with or without the help of a pump.

With the help of the extracorporeal circulation system mentioned above as an example, blood or blood plasma is led through the adsorption system from a patient via an accordingly cannulized blood vessel. Viruses present in the blood or plasma are adsorbed at the porous particles of the adsorber material and thus removed from the stream before the blood or plasma is reintroduced into the organism. This process can be repeated at certain intervals until the resulting reduction of the viral load leads to an improvement and healing of the infection. A combination with other treatment strategies is possible as well.

With the help of the adsorption system according to the present invention, viruses and viral components are irreversibly removed from the physiological fluids to be treated, i.e. under physiological conditions, the filtered out components are bonded securely to the adsorber material.

Surprisingly, with the help of the adsorption system according to the present invention, viruses and viral components can be removed from various physiological fluids, even blood, with a high degree of efficiency, without requiring a prior separation of cellular blood components, e.g. by filtering the blood to obtain plasma. Even after only passing through the adsorption system once, the virus titer of a blood sample can be reduced by more than 90%.

The adsorption system and the adsorber material of the present invention are excellently suited to remove viruses and viral components from physiological fluids. Examples include representatives of the families Hepadna, Flavi, Corona, Filo, Bunuya, Arena and Retro viridae. In addition to hepatitis C, the present invention should particularly also be used to treat diseases such as e.g. hepatitis B, AIDS or viral hemorrhagic fevers. Other biological materials for whose removal from physiological fluids the adsorption unit and the adsorber material of the present invention can be adjusted include for example RNA fragments, prions, plasmides or other biological nanoparticles.

According to the present invention, the adsorption system and the adsorber material described herein can be used to treat diseases, in particular viral infections, caused by the pathogens listed above. Furthermore, the adsorption system and the adsorber material can be used to prepare a drug/medicinal product for treating these diseases.

The present invention is described in more detail in the following examples; however, they are not intended to restrict the invention in any way.

### PREPARATION EXAMPLE 1

General preparation instructions for spherical particles with a size of about 100 μm and pore radii of about 80 nm

In a heatable reaction vessel, a solution of 0.5 g polyvinyl alcohol with a degree of hydrolysis of 88% and 0.1 g polyvinyl alcohol with a degree of hydrolysis of 98% in 400 ml water is prepared and a mixture of 10 g methyl methacrylate, 10 g ethylene glycol dimethacrylate, 0.5 g AIBN, 10 ml cyclohexanol, and 20 ml octanol is added under stirring. This mixture is then polymerized at 70° C. under stirring until the reaction is completed. The particles are washed several times with water and methanol, treated with methanol and acetone in a Soxhlet extractor, and dried to constant weight at 100° C. in a vacuum.

### PREPARATION EXAMPLE 2

General preparation instructions for spherical particles with a size of about 100 μm and pore radii of about 450 nm

In a heatable reaction vessel, a solution of 0.75 g polyvinyl alcohol with a degree of hydrolysis of 88% and 10 g sodium sulfate in 800 ml water is prepared and a mixture of 10 g methyl methacrylate, 10 g ethylene glycol dimethacrylate, 0.5 g AIBN, 18.3 ml butyl acetate, and 36.6 ml octanol is added under stirring. Then 10 g calcium phosphate are added and the mixture is polymerized at 70° C. under stirring until the reaction is completed. The particles are washed with water and hydrochloric acid and several times with water and methanol, treated with methanol and acetone in a Soxhlet extractor, and dried to constant weight at 100° C. in a vacuum.

### PREPARATION EXAMPLE 3

General preparation instructions for spherical particles with a size of about 100 μm and pore radii of about 130 nm

In a heatable reaction vessel, a solution of 1 g polyvinyl alcohol with a degree of hydrolysis of 88% in 800 ml water is prepared and a mixture of 10 g methyl methacrylate, 10 g ethylene glycol dimethacrylate, 0.5 g AIBN, 18.3 ml toluene, and 36.6 ml octanol is added under stirring. This mixture is then polymerized at 70° C. under stirring until the reaction is completed. The particles are washed several times with water and methanol, treated with methanol and acetone in a Soxhlet extractor, and dried to constant weight at 100° C. in a vacuum.

### PREPARATION EXAMPLE 4

In order to modify the particle size, Examples 1 to 3 are repeated wherein polyvinyl alcohols with molar masses of 30,000 to 200,000 and degrees of hydrolysis of 70% to 98% as well as polyvinyl pyrrolidone with molar masses of 10,000 to 100,000, polyacrylamide, hydroxyethylcellulose, and polyacrylic acid sodium are used as protective colloids in the aqueous phase.

### PREPARATION EXAMPLE 5

In order to modify the particle size, Preparation Examples 1 to 3 are repeated wherein the amount of the aqueous phase is varied between 200 and 800 ml, and the ratio of aqueous to organic phase is varied from 20:1 to 5:1.

### PREPARATION EXAMPLE 6

In order to adjust the particle size, Preparation Examples 1 to 3 are repeated wherein the ratio of monomer to porogenic agent is changed from 2:1 to 1:5.

### PREPARATION EXAMPLE 7

In order to modify the particle size and control the pore morphology, Preparation Examples 1 to 6 are repeated wherein in addition to cyclohexanol and butyl acetate, toluene, cyclohexanone, butanone, xylene, trichloromethane, ethylacetate, linear polymethyl methacrylate, or a mixture of several of these components are also used as the porogen component in the organic phase.

### PREPARATION EXAMPLE 8

In order to modify the particle size and control the pore morphology, Preparation Examples 1 to 6 are repeated wherein in addition to octanol, other primary alcohols as well as alkanes with six and more carbon atoms or mixtures of these components are also used as the porogenic component in the organic phase.

### PREPARATION EXAMPLE 9

In order to improve particle quality and control size distribution, Preparation Examples 4 to 6 are repeated wherein the amount and type of the salt added to the water phase are modified. In addition to sodium sulfate, sodium chloride, calcium chloride, copper chloride, disodium hydrogenphosphate, and sodium acetate, and in addition to calcium phosphate, magnesium carbonate, calcium carbonate and highly disperse silicic acid are used as well. The amount is up to 20 g.

### EXAMPLE 10

Examples 1 to 6 are repeated wherein in addition to methyl methacrylate, ethyl methacrylate, butyl methacrylate, 2-ethylhexyl methacrylate, cyclohexyl methacrylate, phenyl methacrylate, styrene, 2-hydroxyethyl methacrylate, methacrylic acid amide, and 2-vinylpyrrolidone are also used as monomers, and in addition to ethylene glycol dimethacrylate, divinyl benzene, butylene glycol dimethacrylate and mixtures of the above-mentioned components are used as well. This way, the surface characteristics can be modified.

The effectiveness of the adsorption unit according to the present invention against different species of viruses was verified for the Bovine Virusdiarrhoe virus (BVDV) and the hepatitis C virus (HCV).

### EXAMPLE 11

### Determination of Virus Titer

The virus titers of the Bovine Virusdiarrhoe virus (BVDV) were determined by end point dilution method for the infectiousity measurement (virus titration). The calculation of the titer as logCID₅₀/ml (CID₅₀: culture infectious dosage on the basis of 50%) was carried out according to Kaerber (Arch. Exp. Pathol. Pharmakol. 1931: 162, 480).

The virus titers of the hepatitis C virus (HCV) were determined by means of the quantitative HCV detection (unit: IU/ml) using the method of signal amplification by a b-DNA process (branched-DNA process).

### EXAMPLE 12

### Depletion of BVDV from a Virus Suspension

300 mg polymer particles (prepared analogously to Preparation Example 1) with an average pore radius of 81 nm, a porosity of 0.45 and a specific surface of 608 m²/g were suspended in PBS (phosphate-buffered saline). The suspension was filled into column made from plastic material (diameter 1 cm, particle bed height 2.2 cm, particle bed volume 1.7 ml). In order to prevent particles from escaping, the particle bed is closed at the top and at the bottom with a filter cloth having a mesh size of 40 μm. The dead volume of the particle-filled column including the connecting and outlet tubes is 2.5 ml. The particles were conditioned with PBS.

1 ml of virus suspension in PBS with a virus titer (given as logCID₅₀/ml) of 5.8 was pumped with a constant flow of 0.25 ml/min onto the particle-filled column by means of a syringe pump. Then the column was eluted with 5 ml PBS (0.25 ml/min). The eluate (6 ml) was collected. Then the column was washed with 6 ml PBS (0.25 mmin); the wash solution was collected as well. The wash solution and the eluate were frozen at −80° C. to determine the virus titer. A virus suspension diluted 1:6 with PBS (corresponding to the dilution of the virus suspension after eluation from the column) was used as a reference sample.

The virus titers of the reference sample, the eluate and the wash solution were determined analogously to Example 10. The virus titer (logCID₅₀/ml) of the diluted starting suspension was 4.4. No infectiousity could be detected in the eluate and the wash solution.

### EXAMPLE 13

### Virus Depletion from HCV-Positive Plasma

300 mg polymer particles (prepared analogously to Preparation Example 2) with an average pore radius of 427 nm, a porosity of 0.43 and a specific surface of 88 m²/g were filled into columns analogously to Example 11. The particles were conditioned with PBS.

5 ml of a plasma preparation of HCV-positive plasma were pumped with a constant flow of 0.25 ml/min through the particle-filled column by means of a syringe pump. The eluate (5 ml) was collected and frozen at −80° C. until the virus titer was determined. A plasma preparation diluted 1:2 with PBS (corresponding to the dilution of the plasma preparation after eluation from the column) was used as a reference sample.

The virus titers of the eluate and the reference sample were determined analogously to Example 10. The virus titer of the reference sample was 25,910 IU/ml; the virus titer of the eluate was below the quantitative identification limit of 615 IU/ml. This corresponds to a virus depletion of >97%.

### EXAMPLE 14

### Depletion of BVDV from Blood

300 mg polymer particles (prepared analogously to Preparation Example 3) with an average pore radius of 131 nm, a porosity of 0.51 and a specific surface of 105 m²/g were filled into columns analogously to Example 11. Before the experiment was started, the particles were conditioned with Dextran 40 infusion solution.

5 ml of a blood preparation of human hirudin blood to which a BVDV suspension with a high virus titer had been added were pumped with a constant flow of 0.1 ml/min through the particle-filled column by means of a syringe pump. The eluate (5 ml) was centrifuged for 10 min at 2,200 g in order to separate the blood cells and then frozen at −80° C. until the virus titer was determined. A blood preparation diluted 1:2 with Dextran 40 infusion solution (corresponding to the dilution of the blood preparation after eluation from the column) was used as a reference sample.

The virus titers of the excesses recovered from the eluate and the reference sample were determined analogously to Example 10. The virus titer of the reference sample, given in logCID₅₀/ml, was 3.9; the virus titer of the eluate was 2.4. This corresponds to a virus depletion of 97%.

1. Adsorption system for the removal of viruses and viral components from physiological fluids, wherein the adsorption system comprises as an adsorber material particles with an average diameter of 20 to 500 μm having a polymer matrix with throughpores.

2. Adsorption system according to claim 1, wherein the pore radius of the throughpores is between 25 and 1,000 nm.

3. Adsorption system according to claim 1, wherein the porosity of the particles is between 0.25 and 0.75.

4. Adsorption system according to claim 1, wherein the particles have a polymer matrix comprising units derived from methacrylic acid as a monomer.

5. Adsorption system according to claim 4, wherein the polymer matrix is formed by a copolymer comprising methacrylic acid ester and cross-linking agent.

6. Adsorption system according to claim 1, wherein a substance improving hemocompatibiity is applied onto the surface of the particles.

7. Adsorption system according to claim 6, wherein the particles have a polymer matrix comprising units derived from methacrylic acid and the substance improving hemocompatibility is bonded to a polyethyelene glycol chain.

8. Adsorption system according to claim 6, wherein the substance improving hemocompatibility is a polyorganosiloxane.

9. Process for the removal of viruses or viral components from blood or blood plasma, comprising contacting the blood or blood plasma with an adsorption system according to claim 1.

10. Adsorber material comprising particles with an average diameter of 20 to 500 μm having a polymer matrix with through pores for use in an adsorption system according to claim 1.

11. A method of treatment of a viral infection, comprising
a) providing an absorption system according to claim 1, and
b) bringing the adsorption system into contact with blood or blood plasma of a patient using an extracorporeal process.

12. The method according to claim 11, wherein the viral infection is a disease selected from hepatitis C, hepatitis B, AIDS or viral hemorrhagic fevers.

13. A method of treating physiological fluids comprising
a) providing an adsorption system according to claim 1, and
b) bringing the adsorption system into contact with the physiological fluids.

14. The method of claim 13, wherein the physiological fluid is whole blood.

15. The method of claim 13, wherein the physiological fluid is blood plasma.
